(19) United States
(12) Patent Application Publication
(10) Pub. No.: US 2020/0027565 A1
(43) Pub. Date: Jan. 23, 2020

(54) A LAYERED MEDICAL DATA COMPUTER ARCHITECTURE

(71) Applicant: Antoine C.E. Poppe, Kessel-Lo (BE)

(72) Inventor: Antoine C.E. Poppe, Kessel-Lo (BE)

(21) Appl. No.: 16/484,140

(22) PCT Filed: Feb. 20, 2018
(86) PCT No.: PCT/EP2018/054156

§ 371 Date: Aug. 7, 2019

(30) Foreign Application Priority Data
Feb. 21, 2017 (EP) 17157068.2

(51) Int. Cl. G16H 50/70; G16H 50/20

(52) U.S. Cl. CPC G16H 50/70; G16H 50/20

(57) ABSTRACT

The invention relates to the field of medical data handling system, related methods and uses thereof.

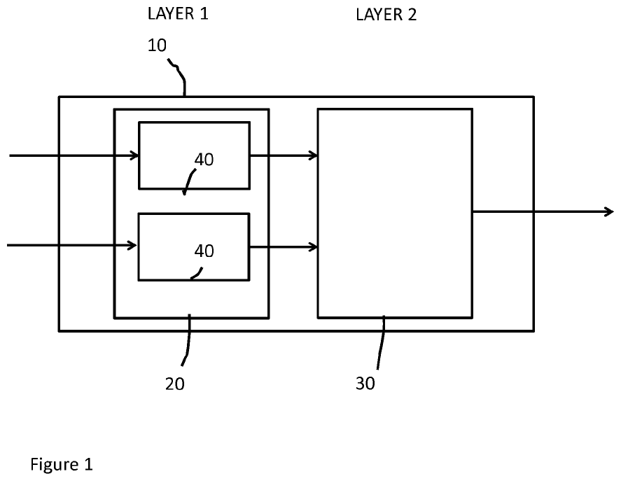

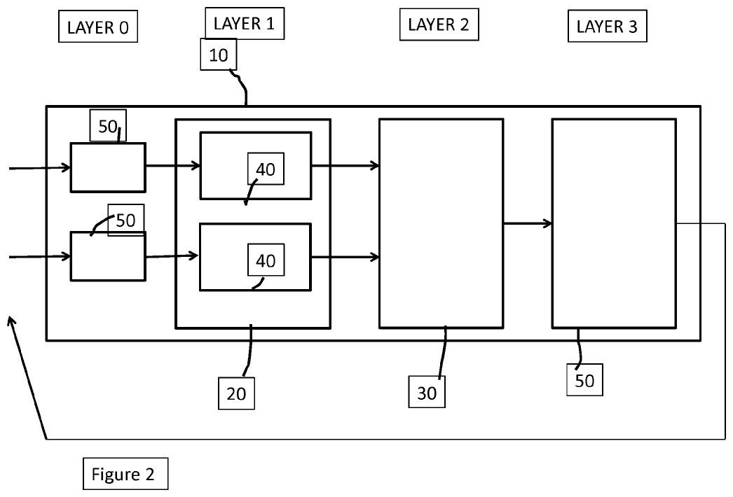

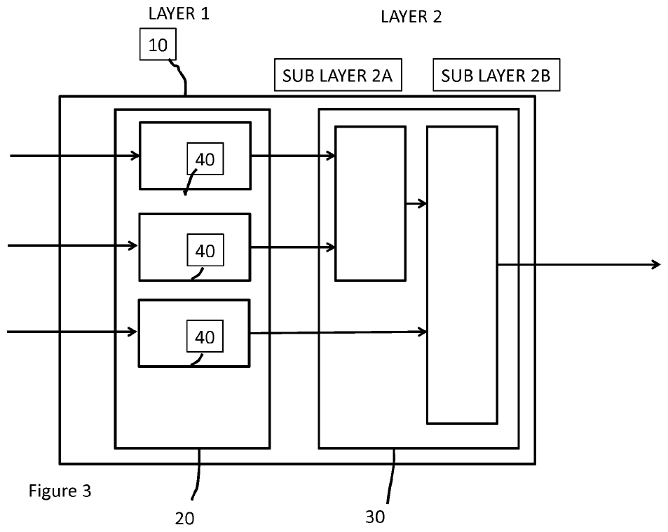

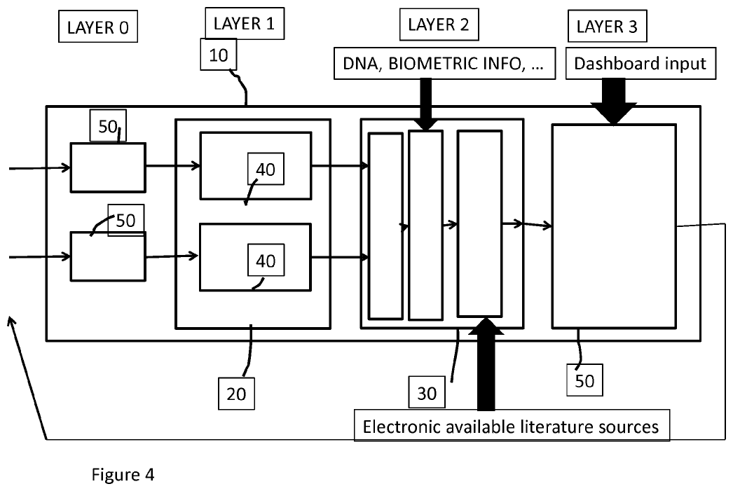

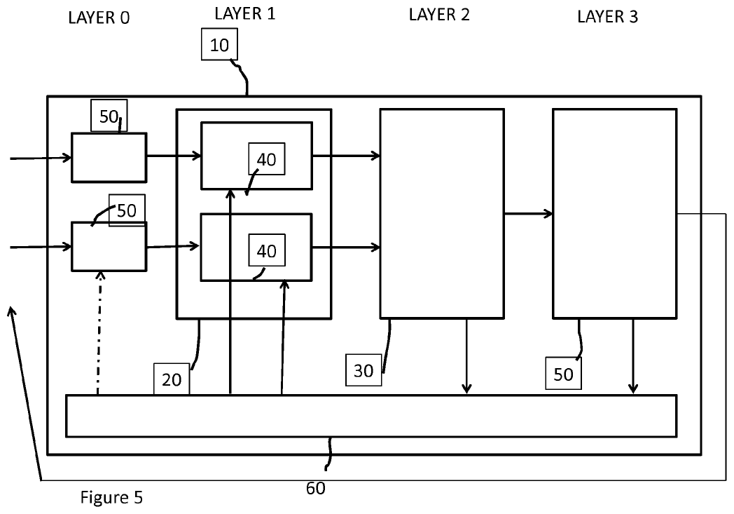

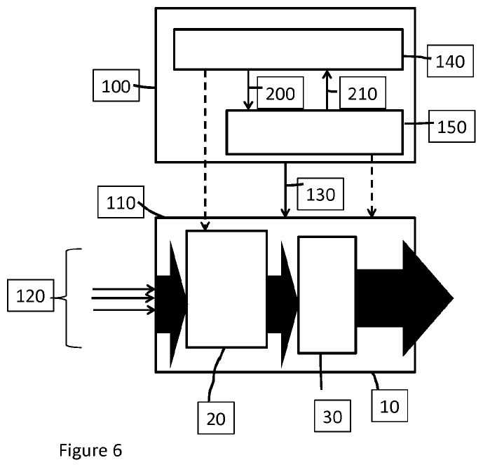

### FIELD OF THE INVENTION

The invention relates to the field of medical data handling system, related methods and uses thereof.

### BACKGROUND OF THE INVENTION

The field of medical data handling is today characterized in that there exist an enormous set of such medical data but organized in silos per discipline, say per sensor (used in the broad sense of the word here as information) available about a human (or animal).

While in principle combining all said sets of different sensor data into one computer system by using of Artificial Intelligence (AI) techniques can be put forward as the way to go, such mere combination is probably in practice hard to achieve while the per discipline build up expertise in medical data analysis and data processing might be difficult re-used in such global approach.

In the field of Artificial Intelligence, Deep Learning is a known technique, however typically used on 2- or more dimensional data such as images.

### AIM OF THE INVENTION

The invention aim at overcoming the above discussed problem and provides a carefully selected workable technical solution (being real-time deployable) with a layered approach, leveraging use of a plurality of diverse medical data bases in combination with a mix of discipline specific data processing algorithms with generic Artificial Intelligence methods, more in particular Machine Learning.

### SUMMARY OF THE INVENTION

The invention relates to the field of (at least partially real-time available) medical data handling (such as data analysis or data processing) systems and/or computer or compute architectures, related use and tuning (learning) methods, uses thereof and related arrangements and tools (such as suited data bases).

For sake of clarity in the further description a medical data computer system is described in the sense of a computer system which has been trained, adapted, tuned based on a plurality of heterogeneous sensorial information of different persons (“learning phase”), and ones being trained, adapted or tuned, will generate monitoring of one or more parameters of a human, representative for his or hers physical condition or state (“use phase”), preferably the obtained information is accurate and/or precise enough to even further generate at least one action (providing monitoring information to a third party and/or generating alarms and/or performing preventive measures) for the environment of said human.

In essence an at least 2 layered medical data computer system (10) or architecture is proposed comprising a first computer subsystem (layer 1) (20); and a (data fusion) second computer subsystem (layer 2) (30), wherein said first computer subsystem, comprising: a plurality of further third computer subsystems (40); each further third computer subsystem being adapted for a single sensor data processing; and said second computer subsystem being adapted for data processing a plurality of the outputs, generated by each of said further third computer subsystems.

The following aspects can be clarified already at this level.

The layer 1 allows for having single sensor data processing which is specific for the type of sensor (or information) at hand and hence enables embedding of (some kind of) related discipline expertise therein. Such sensor data processing can be based on machine learning, but preferably one taking into account also per discipline expertise.

The layer 2 de facto has to be multidisciplinary (as at least two different kinds of information are fed to it) and hence more generic machine learning techniques (albeit embedding potential multi-disciplinary information remains possible). Note that while layer 2 is preferably fed with preprocessed (by layer 1) information, feeding in directly some sensor information remains possible.

At the stage we can highlight that the key layers are disjoint, but they interact with each other in a bi-directional way. Deep learning methods play a crucial role here. Information is propagated from lower to higher layers, while the representation in each layer is determined by the information needs at higher layers.

Besides the data layers discussed above, two special layers can optionally be provided.
Governance & Security is taken care of across all layers to have a total approach. At higher levels, there are more possibilities to detect and control whether confidentiality/privacy has been compromised.
Learning and security, in addition, does not only occur between layers, but also between vertical silo's, in a matrix model, using transfer learning between domains.

Above the two core layers (specific, less specific) are described. A pre-processing layer (layer 0) (50) for generic conditioning sensors signals (like amplifying, filtering, . . . ) can be placed before layer 1 when felt necessary.

In more general terms the at least 2 layered medical data computer system or architecture can be organized in that the (data fusion) second computer subsystem (layer 2) in itself comprises of further computer subsystems, operating a preselected groups of sensors, which are then further combined in a sub-layered approach, moving gradually from the sensor (group) specific approaches to the generic approaches.

In a preferred embodiment for generating the actions described before another layer is added via fourth computer subsystem (40), being capable of computing in real-time at least one action, based on inputs for the second subsystem.

For sake of completeness sensor is used throughout the description in the broad sense of the word as information, available about a human (or animal), particularly including information obtained via typical sensors (such as but not limited to ECG, blood pressure, blood sugar or glucose content, blood oxygen content, irrespectively where those are on the body, under the skin or even (in part) within the body (e.g. in the case of use of nano-sensors) but also advanced systems like lab on chip may provide suitable information for the invention as described. It is worth noting that in essence all of the above sensors (although the invention is not necessary limited thereto) are sensors generating 1 dimensional time series data.

### DETAILED DESCRIPTION OF THE INVENTION

As indicated above the invention relates layered heterogeneous medical data real-time handling computer systems, related methods and uses thereof as further detailed below.

For sake of convenience a particular name to a layer may be given in order to clarify its (core-)task.

In an embodiment of the invention the following allocation of tasks is given to the layers.
1. Monitoring & infrastructure layer, silo-based, possible use of AI per silo(=module) with either unsupervised and supervised learning.
2. Integration layer, (semantic) interoperability between modules. Giving meaning to raw data collected in Layer 1. Meaning is not only constructed bottom-up, but also top-down. Using both unsupervised and supervised Deep Learning
3. Analytics & Diagnosis: insights, alerts, . . .
4. Advice layer: recommendations to patient & modules
5. Human/CPS Interaction layer: where patient & caretakers interact with the AI; learning from the AI as well as teaching the AI!
6. Social layer: interaction with other patient agents; e.g. communities, government initiatives,
7. Policy & research layer: prevention strategies, driven by bottom-up data. Automated clinical trials in the background (using RCT).

Those systems are suitable for preventive medical monitoring, online, in real time, wireless with automatic alarming and location of the patient, optimal recuperation and consequential medical treatment for all people or tuned for a selected target group like sport or top sport people.

Those systems are further suitable in completely closed systems for online monitoring and administering, including permanent follow-up and potential correction of clinical test on humans for the new pharmaceuticals. It is worth noting that for such purpose feeding in as so-called sensor information further information related to the administering of a drug (which might be based also on a real sensor but also just based on a console) is preferred.

Those systems are suitable for use in combination with vehicles such as in cars or planes, online, in real time, wireless with automatic alarming, location and safe stopping of a car when medical problem makes further safe driving of a car, flying a plane, impossible and dangerous.

The combination of the real-time health measuring with the multi database training actually results in that a high performance real time health measurements approach is complemented with already registered health data on health monitoring platforms, increasing the correctness level of the medical diagnosis and treatment proposal.

The above systems can be deployed in various manners and in a preferred embodiment an embedded personal assistant plug-in for personal dialogue robot and for personal interaction and input (speech-to-text) is used in combination with our platform, including all kinds of chat bots to provide personal feelings about healing progress of his/her disease to a patient

The invention can be complemented with add-ons (possibly per application) and other supportive complementary and embedded structural support systems.

As exemplary embodiments the central monitoring platform can be connected with Al driven virtual voice agents/robots located in the home, the car, as app on smart phone or satellite phone.

As exemplary embodiments the invention can be provided with systems for optimal linking to databases for latest info for correct diagnosis, medicines and optimal additional treatments of disease of a patient.

As exemplary embodiments the invention can be provided with search engine for gradually building up of regional, national and international community of people with same diseases (community building).

As exemplary embodiments the invention can be provided with voice generated communication module between patient, medics and family, like chat bots, with possibility for additional medical data import.

As exemplary embodiments the invention can be provided with automatic and secured payment module from patient to medical actor plus automatic refunding module from health insurance company to patient and/or medical actor.

As exemplary embodiments the invention can be provided with top security modules for medical data transfers and storage as well as for all financial transactions, like, among others, the use of blockchain technologies

As exemplary embodiments the invention can be provided with integrated linkage with personal DNA string and automatic generation through learning algorithms of eventual correction in all medical actions between patient and medical actors: final confirmation of correct diagnosis and best up to date medicinal treatment, preventive medical actions to be taken in the future.

Preferably a cloud-connected operation is ensured.

Due to the fact that devices that monitor physiological data from the patient do not contain large processing power, the learning algorithms will be constraint on processing power. In most scenarios Deep learning (DL) would not be suitable for this challenge. However, we are witnessing more and more DL algorithms being implemented into systems that are constraint on processing power. Furthermore, applying DL algorithms in this layer is also beneficial in the following layers due to the ensemble characteristics of Deep Learning algorithms.

Current state-of-the-art DL technology has been used more and more in embedded systems and have a great potential to be embedded into monitoring devices such as ECG, Glucose, weight, blood pressure, etc.

Initially, each monitoring device needs to be learned offline. Offline means that the data from many patients need to be recorded and stored in a centralized database. This has to happen for each monitoring device that will be used in this system. This can be achieved by storing the data locally in each user device and with the consent of the user send it to the cloud.

In the cloud is assumed that the computational power is not a limitation. As a result, we can train a large Deep learning network on the collected dataset until we are sure we have a reliable accuracy.

Online Embedding

The goal of this phase is to embed the learning algorithms in the monitoring devices. In order to do this some neurons of the DL algorithm need to be pruned. This is done by using the state-of the art pruning methods for DL algorithms.

At the end of this process the DL algorithm is “cropped” onto a size that can be feed on a monitoring device. When the DL is inserted, we change the state from Offline to online. This means that the algorithm will keep learning and predicting of the user. This prediction can be used in further layers to predict patient's future trends.

Due to the fact that we dramatically “cropped” the neurons of the DL algorithms, online learning could potentially worsen the reliable accuracy of the DL that was learned offline. As an optional method we will connect the monitoring devices via WIFI or Bluetooth to devices that can hold more data and have more processing power such as mobile phones, cloud computing. This way we can keep the size of the network big enough with the hope of improvement in the future via online learning.

The above described possible embedding of the invention in larger systems also puts forwards some features of the invention.

The closed loop aspect or support of the system via the sensors on the person (and potential patient) over the layered data processing towards generation of actions suitable for the environment (or actors therein like equipment up to robots) wherein such person resides must be stressed.

Further the generality of its architecture enables target group tuning, for instance by use of suitable reference databases to train the various layers.

Integration Layer:

Due to the use of DL in the previous layers, we can merge the data of every device together to make higher level predictions about a subject's health such as diagnosis, optimal treatments and procedures to follow.

The DL algorithm is composed of layers. Each layer encodes higher levels of information from the raw data. In this layer we will gather all the higher layers of each DL-monitor devices and join it together into a single network. This network will now have enough information to make higher order decisions such as make a diagnosis or give health advice to users.

Additionally, we could have a bidirectional flow to upper layers by making use of transfer learning in DL. This means that we can use information of other monitoring devices to increase the accuracy of another monitoring device. Therefore DL allows us to take full advantage of the raw data.

As indicated above the term sensor information is to be understood broadly and for instance inputting DNA information of a person should also be considered as a (permanent) sensor input.

Further the layered architecture leverages on real-time health measuring and the existence of multiple databases and actually increases their usage.

To ensure sufficient security, in an embodiment of the invention (substantially) all critical medical data applications and communication connections will be secured, preferably with a BLOCKCHAIN-enabled (or any similar solution) approach for assuring and realizing an absolute and total security. This can (in combination with other measures) be applied for

1. Supporting the cloud storage network

2. all IoT applications and integrations on the invented core platform

3. the healthcare medical data storage, transfer, access, archiving

4. payment and remuneration embedded and/or complementary systems.

In essence the invented computer system while being layered may provide for security measures (like encryption) amongst one or more it of its layers. Note that while we describe here an entirely computer system (with its variety of subsystems) does not limited the invention to at one physically location present hardware system. In an embodiment of the invention the proposed system is implemented in a distributed fashion and (besides security amongst layers) also security between the computation nodes involved might be provided.

Finally as indicated above the computer system is suited to deploy monitoring applications, more in particular provides support to generate alarms, location of the human in danger to thereby support operations to save him or her. However while the above might suggest a rather inactive role of the system, the computer system is suited also to implement a more active approach

wherein, in case no response is occurring based on the alarms and the provided information, the system might invoke a further Al approach for identifying (via an intelligent search) alternative support operations, alarming those and insisting on them to help the patient based on the data provided by the system to them.

The multi layered and/or deep learning methods, algorithms and/or platform structure, adapted for use on health databases are further operated through personalized and secured dashboards communicating through online and in real time means of communication on a per operator-user basis, allowing besides passive (warning based) prevention, pro-active health self-control and (co-)management.

Human/CPS Interaction Layer

In this layer we can use the latest trend of DL called Deep Reinforcement learning (DeepRL). These state-of-the-art methods currently combine 2 of the most powerful AI algorithms: Deep learning and Reinforcement learning. We have previously seen that DL allows us to model a complex system just using its inputs and the outputs. However, with DL alone we will never be able to do better than underline model of the complex system. As an example, let us assume that we could gather a great amount of diagnosis from doctors all over the world. Then we can create a DL model from the diagnosis data by giving the DL algorithm the inputs of the symptoms and its diagnosis. This will result in a DL system that can predict the diagnosis of a patient as good as the best doctors in the world but not better. In order to become better, we need to use reinforcement learning (RL). This Al algorithm will try to find solutions to a problem by trial and error. After several trials, it is expected that the RL algorithm will eventually come up with better diagnosis.

However, the RL trials normally take very long time and it can also be very dangerous for the patients. To solve this, new research has come up with a Deep RL algorithm that can learn from demonstration. This means that the doctors will teach the Deep RL algorithm how to come up with diagnosis, at the same time the Deep RL can suggest to the doctor another diagnosis if the system “beliefs” it would be better.

While above the various data processing layers are addressed in more detail, now the further functional layers, implementable on top thereof, are further discussed, with an illustration on the further specific mapping on the above described system.

Recall that a first functional layer relates to prevention in the background and in essence is a basic combination of various sub layers, in particular (i) one for initial and provisional diagnosis, (ii) one for activation of the medical and other actors involved aiming at immediately correct diagnosis, treatment proposal and effective actions undertaken by the responsible medical and personal actors.

Recall that this functional layer is extremely suited for mapping on the deep learning algorithm per stand-alone physiological data silo's of the patient, originating from monitoring devices for ECG, glucose, weight, blood pressure etc and all personal health data originating from health consultations at your personal doctor, health specialists and clinic visits and stays and any other medical data input by yourself and/or any authorized third party through a connected dashboard.

In essence the autonomy of this layer—in that without any action needed by any actor (except when one of your medical actors wants to connect with your health monitoring devices for regular control and/or of recovery) but with alarm and localization of the patient in case of an imminent health problem arising based on incoming health data which are reaching predefined critical tunnel (maxima and/or minima) values.

Another additional layer of intelligent agent is combining all AI agents of the stand-alone physiological data silo's of the patient in order to generate an initial and provisional (a) diagnosis, (b) optimal treatments and procedures to follow, (c) best first trial medication mix and (d) medical treatment time line and follow up procedures with eventually and additionally putting forward and/or suggesting (i) additional medical and/or pharmaceutical questions to answer, (ii) new medical measurements to be taken, (iii) suggested consultations and connections with additional proper and/or third party AI Agents and finally generating all those provisional findings as urgent advice and eventual actions to be taken by each respective curative party involved, and findings which will appear online and in real time as a patient, through your proper dashboard on your personal smart phone, tablet etc receiving through your personalized HEALTH ANGEL APP or through their proper dashboard on the smart phone, tablet, computer screen of all medical actors involved and authorized to assist the patient concerned, with accompanying request(s) for immediate actions to be taken like organizing an ambulance, search and final appointment for examination of the best specialist(s) and eventually consequential admission to the clinic on the specific dashboard of the smart phone, tablet or computer screen of authorized family and friends being linked to the interconnected HEALTH ANGEL network. The automatic safe data storage and payments for medical actors and consequential prompt refunding by health insurance companies through a block chain secured app complemented for correct payment eventually using a Med coin kind of crypto money.

A further additional layer relates to complementary and supplementary controlling and optimal matching of provisional diagnosis findings with the deep learning algorithm which interprets our personal genome data DNA with consequentially corrections, adaptations and complementary information and treatment indications. These complementary deep learning algorithms can build up an extremely large knowledge about negative health indications through biometric face recognition and continuous automatic health evaluation. The biometric face recognition will be as far specialized in number of face coverage points, color and heat sensors that this layer will become able to salute the user anytime about his/her health condition. In a similar way, voice analysis, personal fluids analysis through a personal lab on a chip, can complement all above findings about the physical and physiological situation of the patient. Complementing and controlling of initial diagnosis and medical treatment(s) based on the previous layers through different third parties Intelligent Agents, specialized in additional and/or complementary health diagnosis and treatment knowledge, in order to come to the final and definitive medical decisions about the best diagnosis and treatment for the patient concerned.

A further functional layer relates to third parties top visions input: based on our medical history, this deep learning algorithm of this layer, starting and based on your personal medical history and genome DNA, our proper Al agents of previous layers scout automatically, autonomously and permanently third party sources of the most recent top medical information sources worldwide so that, when called upon when the patient enters into an alarm phase, the provisional diagnosis, treatment and medicine mix, are completed and eventually corrected through this additional layer, so that final decisions can be made based on the best global findings of that moment. A permanently linked input could for instance come from OntoForce which continuously updates her database with the most complete diagnostic symptoms and latest medical treatment and medicine mix for all known and recently detected diseases. Another linked input can come from various Al automated diagnosis engines and consequential medical treatment advisory engines. They can control our proper Al solutions in a complementary and supplementary way.

In a further functional layer a total and all-round security layer for medical data, payments and reimbursements are provided. While at the start of the opening of your personal dashboard on your smartphone will happen through Biometric face recognition through the front camera of the smart phone, fraud will be excluded at the start. Simultaneously, a next generation Biometric face recognition (based on the scanning of millions of points, color variations, forms, eyes etc) will generate a first health evaluation of the person concerned with or without any additional personal data input of the patient himself. Additionally a complete security layer will be developed for the totality of our hard and software solutions and all our proper and third parties integrated deep learning layers. A Lansweeper platform will secure our complete network system and prevent, destroy and restore any impact of cyber attacks, ransom ware outbreaks or any other general and/or specifically targeted cyber attack on our complete structure, while in case of any damage, reparation will take place instantly through built-in self repairing programs. Block chain will realize the complete medical data security and privacy as well as the absolute operational stability and security of all devices, networks, platforms and databases including the IoT part of our total solution. We will include Biometric facial recognition and time stamping of all communications and/or data exchange between all actors involved in our complete multi layered structure. All dashboards of all communication platforms will have all secured

Identification hard and software features through Lansweeper, Block chain and biometric facial recognition and time stamping. Our general security layer should also include a layer of adversarial defenses into our deep neural network applications like the regularly testing through generative adversarial networks (GANs). Models to be used are based on AI deep learning algorithms which will detect and destroy adversarial attacks.

Provided with the above one or more (functional) layers, even further layers relating to pro-active and preventive personal health management initiative, can in embodiments of the invention, be added.

Instead of waiting for the automatic preventive monitoring as described above, the same platform can be activated by the patient him/herself, by any medical actor or authorized family member or friend. In this case by personal initiative health measurements of the patients are being taken and a request for the whole automatic diagnosis through the various deep learning algorithms layers is started. This procedure can be advisable when the patient is not feeling well and/or when a third party like family, friends or a medical actor advice to do so.

The above will also apply for the same combination of all components of our multi layered structure of deep learning algorithms but then specialized for one or a combination of several diseases like a unique app exclusively for heart patients, for diabetics, obesity patients, patients with high blood pressure, COPD patients etc.

This will also apply when our multi-layered structure of deep learning algorithms is partly and/or completely applied in other fields of application like, but not limited to, monitoring clinical test on humans, sport and top sport applications, medical coaching and advisory applications

Provided with the above one or more (functional) layers, even further layers are foreseen, relating to unique interconnectivity solutions between Deep Learning Algorithm layers and human actors involved by developing and using customized dashboards. In order to realize an always-on top secured absolutely personalized interconnectivity and easy operational communication between all building blocks, parties and persons involved, unique open and become operational for each user on his/her personal smart phones, tablets, computer screens only after identification through our

Unique Biometric face recognition will connect and/or instantly link the medical operator to all data and info needed for his/her treatment of their patient(s) concerned. The top secured interconnection through and between the specialized dashboard per layer will be organized in a multidirectional communication line so that the final diagnosis, total treatment schedule and timeline (of medicines mix and timing, stay and treatment-operation in clinic,) can only start and being activated after the final OK of the authorized medical actor(s) involved. That final go, together with all medical data which served for this final decision of the medics concerned should be notified and time stamped by each medical actor on the medical record of the patients.

This will facilitate in case later disputes should arise concerning those medical decisions

In yet another further functional layer based on specifically adapted deep learning algorithms, a special dashboard interface is provided with all robots assisting robots of a human being: this can be a robot for assisted living, assisted or autonomic car driving, medical robot assistants as well as any field were robots will assist the human being, will be integrated in our totally integrated health app.

In summary the above describes the ambition level of various layers to be deployed on the claimed invented computer system, which the real-time capabilities (after training, learning or other forms of automated tuning) demanded here are to be emphasized as these lead to the specific selected structure as defined by the input-output and interconnection structure proposed and the function of each layer/subsystem and the elected data processing technique. While the underlying hardware architecture computer system will comprise of one or more general purpose processing elements, (related) memory components, input means adapted for inputting sensorial signals and output means for outputting computed signals and related communication means for those, no particular mapping is proposed in that a central or decentralized system can be used. It is worth indicating that while in discussing computer architectures use of a stack of various layers of abstractions is known and standardized, however contrary to that, here the handling of those various abstraction levels is of an entirely different nature, in that it is the gradual increase of complexity happens along the data flow itself while the imposed signal definitions are new and specific to the field addressed here also. The invention hence entails a computer-implemented method for handling time series data in a predictive model, embedded in the system described above, the method comprising performing historical training of the predictive model by accessing data (representing operational characteristics or measurements) obtained from the real world of humans or animals.

### SPECIFIC EMBODIMENTS OF THE INVENTION

Layered Versus Deep Learning

The provided medical data computer system is designed for combining information from a multitude of different (medical) sources in order to provide diagnostic/remedial information to the user, in particular a layered structure, for instance with a first and second subsystem and optionally a fourth subsystem connected to each other in a particular way are provided, whereby artificial Intelligence techniques such as but not limited to Deep Learning Networks are used in one or more of said layers, preferably for the higher layers. In a preferred embodiment the combining layer (second subsystem) exploits Deep Learning Networks.

While a deep learning platform in itself is also a layered structured and while use of deep learning platforms in one or more of the layered is claimed here, it is worth pointing out here that the invented layered platform is not a deep learning platform on its own as contrary to deep learning platforms wherein in its layers uninterpretable features are formed via the learning, in the invented layered platform exactly known complex features are imposed at each node between said subsystems.

Hence in an embodiment of the invention is provided a medical data computer system (10) comprising a first computer subsystem (20); and a (data fusion) second computer subsystem (30), wherein said first computer subsystem, comprising: a plurality of further third computer subsystems (40); each further third computer subsystem being adapted for a single sensor data processing; and said second computer subsystem being adapted for data processing a plurality of the outputs, generated by each of said further third computer subsystems, wherein said outputs represent one or more (complex) features of said single sensor data in a predetermined format. The same can be said about the interface between the second computer subsystem and the fourth subsystem, hence the output of said second computer subsystem represent one or more (complex) features of the fused sensor data in a predetermined format, which is especially important if said second computer system is using a deep learning technique. For completeness, the above can be extrapolated to the interface between each additional sub computer systems (layers).

Layered-Bidirectional Communication

The description of the medical data computer system (10) as comprising of a first computer subsystem (20); and a (data fusion) second computer subsystem (30) and said second computer subsystem being adapted for data processing a plurality of the outputs (of said first computer subsystem) illustrates a bottom-up (from lowest to higher layers) data communication in one direction. The same can be said for the more detailed embodiments with sensor node processing and the use of additional layers. It is however worth noting that (besides the learning within the layers which require feedback within the layer) the overall governance or control or management of the entirely layered medical data computer system entail also feedback between the layers, and hence a bidirectional communication exist—one direction on the level of the data flow—and another direction based on the feedback flow.

It is worth distinguishing the ‘use’ method (110) for processing data, from a plurality of sensors (120), for a single human (or animal) by the trained layered computer system from the ‘training’ or ‘learning’ method (100), providing parameters (130) (such as the weights of the deep learning networks used), based on processing data, from the same plurality of sensors, but then on a representative population of the target group, to which such single human (or animal) may belong. When the big arrow illustrate the data flow from left to right, more details on the ‘training’ or ‘learning’ method (100) are represented by sub-methods (140), (150), each providing parameters of one particular layer (20) or (30) of the layered computer system (10). A logical sequence of training (illustrated by (200), e.g. referring to a trigger signal) is to execute step (140) (related to layer (20)) first and thereafter followed by executing step (150) (related to the next layer (30)), the above discussion introduces also the reverse sequence (illustrated by (210)). It is for such purpose that the computer system is provided with an overall control system (e.g. a state machine) (60) of said data computer system, suited for supporting the above (cross layer) training method.

Machine Learning at the Sensor Devices

As indicated a (heterogeneous sensor handling) system, comprising a layered computer system and a plurality of sensors (of a different kind or type), each of said sensors being (wired and/or wireless) connected to the layered computer system, whereby said layered computer system is embedded with Artificial Intelligence (Machine Learning, especially Deep Learning) capabilities at one or more of its layers. Given that in a preferred embodiment the first layered is sensor specific, and in order to create communication benefits, part of data processing in the first layer, even if based on Artificial Intelligence (Machine Learning, especially Deep Learning) capabilities, can be shifted into the sensors or monitoring devices if a few considerations are taken into account. In essence in such embodiment a (heterogeneous sensor handling) system, comprising a layered computer system and a plurality of sensors (of a different kind or type), each of said sensors being (wired and/or wireless) connected to the layered computer system, whereby besides said layered computer system, also (part of) said sensors are provided with Artificial Intelligence (Machine Learning, especially Deep Learning) capabilities.

As sensor or monitoring devices that monitor physiological data from the patient do not contain large processing power, the learning algorithms will be constraint on processing power, hence DL algorithms being implementable into systems that are constraint on processing power are to be selected.

Note that applying DL algorithms in this sensor layer or the first layer is beneficial for the following layers due to the ensemble characteristics of Deep Learning algorithms.

In an embodiment of the invention one or more of the sensor devices are (initially) off-line trained.

In an embodiment of the invention one or more of the sensor devices are (optionally after an off-line training) further being on-line trained, preferably the DL algorithm used now is a pruned version of the DL algorithm used in the off-line training.

Optionally temporally or otherwise mixing off-line/online training is used.

1. A medical data processing computer system (10) comprising a first computer subsystem (20); and a second computer subsystem (30), wherein said first computer subsystem, comprising: a plurality of further third computer subsystems (40); each further third computer subsystem being adapted for a single sensor data processing; and said second computer subsystem being adapted for joint data (data fusion) processing a plurality of the outputs, generated by said further third computer subsystems, whereby at least part of said second computer subsystem being further adapted for artificial intelligence based data processing.

2. The computer system of claim 1, whereby at least one (and optionally essentially all) of said further third computer subsystems being further adapted for artificial intelligence (machine learning-)based data processing.

3. The computer system of claim 1, whereby said artificial intelligence based data processing being machine learning based data processing (such as neural networks (NN) and support vector machines (SVM)).

4. The computer system of claim 3, wherein said machine learning based data processing being deep learning based data processing, in particular artificial (multi-layer) neural networks, preferably the machine learning is a classifier.

5. The computer system of claim 1 being adapted in that one or more of said subsystems provide (as part of an overall control system (60) of said data computer system) (feedback) control signals to other subsystems (lower in the layers).

6. The computer system of claim 1, provided in a (heterogeneous sensor handling) system which further comprises a plurality of sensors (of a different kind or type) (being part of a health monitoring platform), each of said sensors separately being (wired and/or wireless) connected to one of said further third computer sub systems, whereby optionally one or more of said sensors also have (some) artificial intelligence (machine learning-)based data processing.

7. The computer system of claim 6, wherein at least part of said sensors (such as but not limited to ECG, blood pressure, blood sugar or glucose content, blood oxygen content sensors) being adapted for monitoring a parameter of a human or animal, preferably at least part of said sensors are on or implanted in the body of said human or animal.

8. The computer system of claim 6, further comprising a fourth sub computer system (50), being capable of computing in real-time at least one action (such as providing monitoring information and/or generating alarms and/or performing preventive measures) for the environment of said human or animal, based on the output(s) of said second computer subsystem, optionally said a fourth sub computer system (40) is adapted for inputting dashboard information.

9. The computer system of claim 8, whereby (part of) said fourth sub computer system (50) being further adapted for artificial intelligence (machine learning-) based data processing.

10. The computer system of claim 6 further comprising (e.g. as part of subsystem (30)) comprises one or more additional sub computer systems (layers), inputting personal info like genome data and/or biometric info; and/or alternatively electronically available literature sources, to perform further data enhancement operations.

11. The computer system of claim 10, whereby at least one (and optionally essentially all) of said one or more additional computer subsystems are further adapted for artificial intelligence (machine learning-) based data processing.

12. A computer system comprising one or more general purpose processing elements, (related) memory components, input means adapted for inputting sensorial signals and output means for outputting computed signals; computer system being adapted for (i) executing a plurality of single sensor data processing methods, (ii) a further (joint) data processing (data fusion) method on a plurality of the outputs, generated by each of said single sensor data processing methods, and (iii) methods for computing output signals based on the outcome of said (joint) data processing (data fusion) method, whereby at least part of said further (joint) data processing (data fusion) method (and optionally one or more or essentially all of said single sensor data processing methods being artificial intelligence (machine learning) based data processing.

13. The computer system of claim 11, wherein said artificial intelligence based data processing is being deep learning based data processing.

14. A method of real-time health measuring which comprises applying a medical data processing computer system (10) comprising a first computer subsystem (20); and a second computer subsystem (30), wherein said first computer subsystem, comprising: a plurality of further third computer subsystems (40); each further third computer subsystem being adapted for a single sensor data processing; and said second computer subsystem being adapted for joint data (data fusion) processing a plurality of the outputs, generated by said further third computer subsystems, whereby at least part of said second computer subsystem being further adapted for artificial intelligence based data processing or a computer system comprising one or more general purpose processing elements, (related) memory components, input means adapted for inputting sensorial signals and output means for outputting computed signals; computer system being adapted for (i) executing a plurality of single sensor data processing methods, (ii) a further (joint) data processing (data fusion) method on a plurality of the outputs, generated by each of said single sensor data processing methods, and (iii) methods for computing output signals based on the outcome of said (joint) data processing (data fusion) method, whereby at least part of said further (joint) data processing (data fusion) method (and optionally one or more or essentially all of said single sensor data processing methods being artificial intelligence (machine learning) based data processing to process data, obtained from a plurality of sensors, of which at least part of said sensors are adapted for (real-time) monitoring of a parameter of a human or animal.

15. The method of claim 14, which comprises generating at least (in real-time) one action for the environment of said human or animal, based on the output of said computer system.

16. The method of claim 14, which comprises determining (when occurring) at least one physical problem of said human and/or animal from (a combination of) a plurality of sensor data, preferably determining (when occurring) a plurality of distinct physical problems.

17. The method of claim 14, which is a method for processing data, from a plurality of sensors, said method comprises the step of executing plurality of single sensor data processing methods and a further (joint) data processing (data fusion) method on a plurality of the outputs, generated by each of said single sensor data processing methods, at least one of said data processing method being artificial intelligence (machine learning) based data processing.

18. A non-transitory machine-readable storage medium storing a computer program product, operable on a processing engine, for executing any of the steps of the method of claim 17.

19. (canceled)

20. A medical data database, compiled to be operable on a computer environment and adapted for being controlled accessible by the computer system of claim 1 comprising, for a plurality of users, a plurality of records, of which part are suited for storage of medical data of such user, each of said records being associated with a different type of sensor.

21. The medical data computer system (10) of claim 1 wherein the system is operably connected to a medical data database compiled to be operable on a computer environment and adapted for being accessible by said medical data computer system (10), comprising, for a plurality of users, a plurality of records, of which part are suited for storage of medical data of such user, each of said records being associated with a different type of sensor.
